(19) United States
(12) Patent Application Publication
(10) Pub. No.: US 2009/0123555 A1
(43) Pub. Date: May 14, 2009

(54) Stimulateable Polymer Particles Exhibiting Reactive Functions, Method for the Production and the Use Thereof

(75) Inventors: Valerie Sabaut-Heroguez, Merignac (FR); Damien Quemener, Castillon La Bataille (FR); Marie-Christine Durrieu, Villenave D'Ornon (FR)

Correspondence Address: YOUNG & THOMPSON, 209 Madison Street, Suite 500, ALEXANDRIA, VA, 22314, US

(73) Assignees: CENTRE NATIONAL DE LA RECHER CHE SCIENTIFIQUE, Paris Cedex 16 (FR); INSERM INSTITUT NATIONAL DE LA SANTE ET DE LA SANT, Paris Cedex 13 (FR); UNIVERSITE DE BORDEAUX I, Talence Cedex (FR); ECOLE NATIONALE SUPERIEURE DE CHIMIE ET DE PHYSIQU, Pessac Cedex (FR)

(21) Appl. No.: 11/630,118

(22) PCT Filed: Jun. 21, 2005
(86) PCT No.: PCT/FR05/01546

§ 371 Date: Mar. 20, 2007

(30) Foreign Application Priority Data
Jun. 21, 2004 (FR) 0406707

(51) Int. Cl. A61K 8/02; A61K 9/14; A61K 31/405; A61K 38/18; C07C 59/62; C07D 209/12; C08F 134/00; C08F 220/28

(52) U.S. Cl. 424/501; 424/401; 514/420; 514/12; 562/503; 548/493; 526/259; 526/320; 526/318.3

(57) ABSTRACT

The invention relates to spherical particles formed by polymer chains containing approximately from 30 to 10000 monomer units derived from monocyclic polycyclic alkene polymerization, wherein at least one monomer unit is substituted by an R chain including ethylene polyoxide which is optionally covalently linked to the polymer units through a hydrolyzable bridge and substituted by a reactive function, optionally engaged in a link with an active principle or a biological molecule such as protein, wherein the chain R is covalently linked to the monomer units. The use of the inventive spherical particles for preparing pharmaceutical and cosmetic compositions or surface coatings is also disclosed.

The present invention relates to polymer particles, and more specifically polymer nanoparticles, which are stimulable, namely which are sensitive to an external stimuli such as a variation in pH or in temperature, exhibiting reactive functions, especially of the type of acid, amine, alcohol or acid chloride, at the periphery, as well as a method of synthesis thereof in one step, and uses thereof.

Stimulable polymers have already been described, which exhibit reactive functions obtained by encapsulation or adsorption of the active ingredients directly in the material or in beads which are themselves adsorbed or grafted on the material.

However, adsorption does not allow a controlled release of the active ingredient. As regards encapsulation, when it can allow, on the one hand, a controlled release of the active ingredient, on the other hand, it proves incompatible with prolonged use and/or when the material is subjected to high stresses (flux, friction . . . ).

Reactive polymer nanoparticles obtained by covalent grafting of active ingredients on the functionalised nanoparticle have also already been described. However, the synthesis of such nanoparticles takes place in two steps (synthesis of the latex, then reaction with the active ingredient) and therefore without direct control of the grafting (random number of functions introduced). Moreover, these nanoparticles do not further possess the active ingredient and anchoring sites allowing a release at a specific location of the active ingredient. These materials are most often intended for vectorisation or for immunological tests.

The present invention aims at providing new polymer particles having a reactive function, optionally engaged in a bond with an active ingredient or a biological molecule such as a protein, the said reactive function being covalently bonded to the said polymers, these latter being obtained in one single step.

The invention relates to spherical particles having a diameter between 10 nm and 100 μm, said particles being formed by polymer chains containing about 30 to 10000 monomer units, identical or different, derived from the polymerisation of monocyclic alkenes in which the number of carbon atoms constituting the ring is approximately 4 to 12 or polycyclic alkenes in which the total number of carbon atoms constituting the rings is approximately 6 to 20, at least one of the said monomer units being substituted by a chain R comprising an ethylene polyoxide of formula (A) optionally covalently bonded to the said monomer units via a hydrolysable bridge

—(CH₂—CH₂—O)ₙ—X (A)

wherein n represents an integer from approximately 50 to 340, especially from 70 to 200, and X represents an alkyl or alkoxy chain with about 1 to 10 carbon atoms, comprising a reactive function of the OH, halogen, NH₂, C(O)X₁ type, wherein X₁ represents a hydrogen atom, a halogen atom, an OR′ or NHR′ group in which R′ represents a hydrogen atom or a hydrocarbon chain with about 1 to 10 carbon atoms, substituted or unsubstituted, where X represents a group comprising a photosensitive function such as polyenes, the said reactive function being optionally engaged in a bond with an active ingredient or a biological molecule such as a protein, the said chain R being bonded covalently to the said monomer.

The invention relates more specifically to spherical particles as defined above, characterised in that the monomer units are derived from the polymerisation of monocyclic alkenes and are of the following formula (Z1)

═[CH—R₁—CH]═ (Z1)

wherein R₁ represents a hydrocarbon chain with 2 to 10 carbon atoms, saturated or unsaturated, the said monomers being optionally substituted by a chain R, or directly by a group X, as defined above.

The invention relates more specifically to spherical particles as defined above, characterised in that the monocyclic alkenes from which the monomer units are derived are:
cyclobutene leading to a polymer comprising monomer units of formula (Z1a) below:

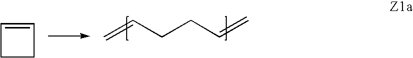

cyclopentene leading to a polymer comprising monomer units of formula (Z1b) below:

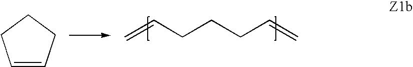

cyclopentadiene leading to a polymer comprising monomer units of formula (Z1c) below:

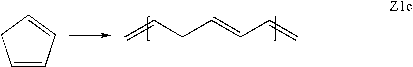

cyclohexene leading to a polymer comprising monomer units of formula (Z1d) below:

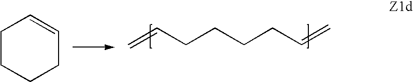

cyclohexadiene leading to a polymer comprising monomer units of formula (Z1e) below:

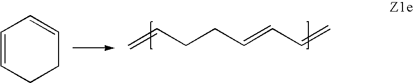

cycloheptene leading to a polymer comprising monomer units of formula (Z1f) below:

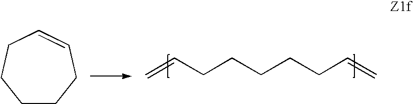

cyclooctene leading to a polymer comprising monomer units of formula (Z1h) below:

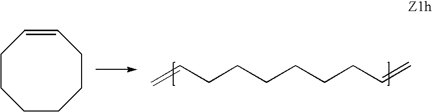

cyclooctapolyene, especially cycloocta-1,5-diene, leading to a polymer comprising monomer units of formula (Z1i) below:

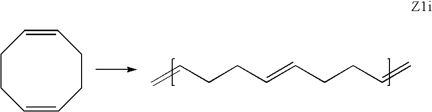

cyclononene leading to a polymer comprising monomer units of formula (Z1j) below:

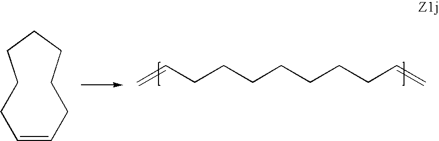

cyclononadiene leading to a polymer comprising monomer units of formula (Z1k) below:

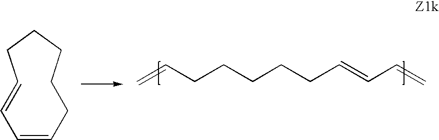

cyclodecene leading to a polymer comprising monomer units of formula (Z1l) below:

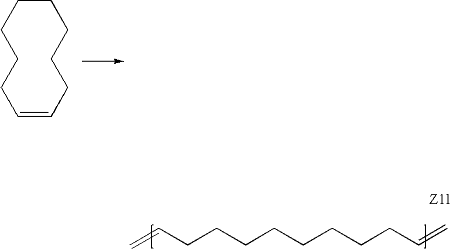

cyclodeca-1,5-diene leading to a polymer comprising monomer units of formula (Z1m) below:

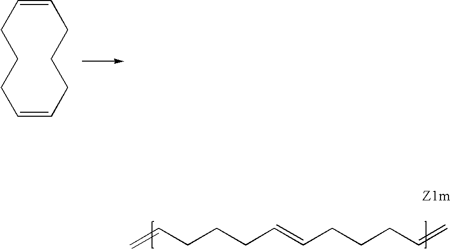

cyclododecene leading to a polymer comprising monomer units of formula (Z1n) below:

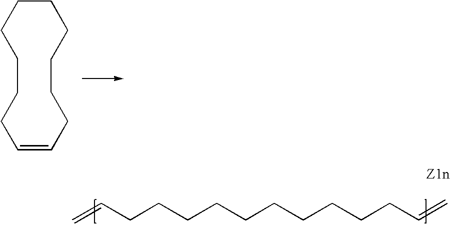

or also 2,3,4,5-tetrahydrooxepin-2-yl acetate, cyclopentadecene, paracyclophane, ferrocenophane.

The invention also relates to spherical particles as defined above, characterised in that the monomer units are derived from the polymerisation of polycyclic alkenes and are:
of formula (Z2) below:

═[CH—R₂—CH]═ (Z2)

wherein R₂ represents:
a ring of formula

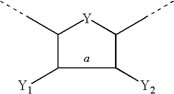

wherein:
Y represents —CH₂—, or a heteroatom, or a —CHR— group, or a —CHX— group, R and X being as defined above,
Y₁ and Y₂, independently of one another, represent H, or a chain R, or a group X, as mentioned above, or form in association with the carbon atoms bearing them a ring with 4 to 8 carbon atoms, this ring being optionally substituted by a chain R or a group X as mentioned above,
a represents a single or double bond,
or a ring of formula

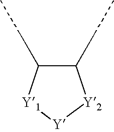

wherein:
Y′ represents —CH₂—, or a heteroatom, or a —CHR— group, or a —CHX— group, R and X being as defined above,
Y′₁ and Y′₂ independently of one another represent —CH₂—, or a —C(O) group, of a —COR group, or a —C—OX group, R and X being as defined above,
of formula (Z3) below:

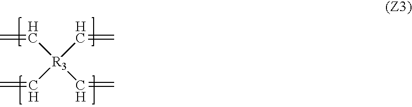

in which R₃ represents:
a ring of formula

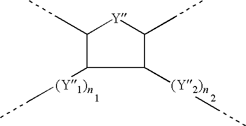

wherein:
n₁ and n₂, independently of one another, represent 0 or 1,
Y″ represents —CH₂—, or a —CHR— group, or a —CHX— group, R and X being as defined above,
Y″₁ and Y″₂ independently of one another represent a hydrocarbon chain with 0 to 10 carbon atoms,
   or a ring of formula

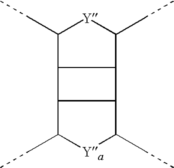

wherein Y″ and Y″a independently of one another represent —CH₂—, or a —CHR— group, or a CHX— group, R and X being as defined above,
or a ring of formula

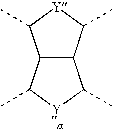

wherein Y″ and Y″a independently of one another represent —CH₂—, or a —CHR— group, or a —CHX— group, R and X being as defined above.

The invention relates more specifically to spherical particles as defined above, characterised in that the polycyclic alkenes from which the monomer units are derived are:
monomers containing a cyclobutene ring leading to a polymer comprising monomer units of formula (Z2a) below:

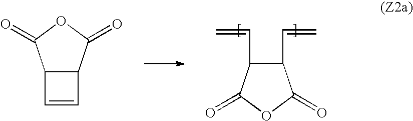

monomers containing a cyclopentene ring leading to a polymer comprising monomer units of formula (Z2b) below:

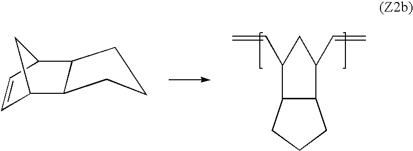

(bicyclo[2.2.1]hept-2-ene)norbornene leading to a polymer comprising monomer units of formula (Z2c) below:

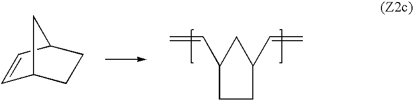

norbornadiene leading to a polymer comprising monomer units of formula (Z2d) below:

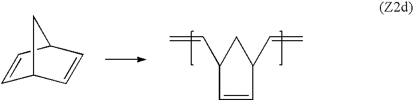

7-oxanorbornene leading to a polymer comprising monomer units of formula (Z2e) below:

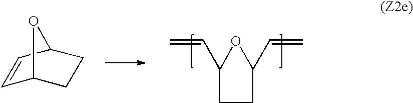

7-oxanorbornadiene leading to a polymer comprising monomer units of formula (Z2f) below:

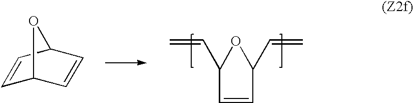

the dimer of norbornadiene leading to a polymer comprising monomer units of formula (Z3a) below:

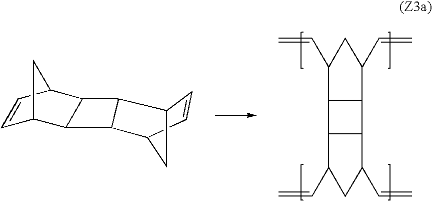

dicyclopentadiene leading to a polymer comprising monomer units of formula (Z3b) below:

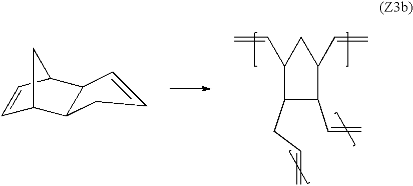

tetracyclododecadiene leading to a polymer comprising monomer units of formula (Z3c) below:

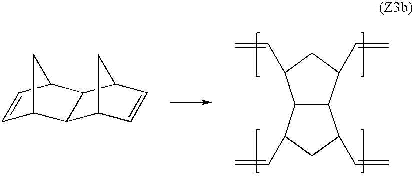

or bicyclo[5.1.0]oct-2-ene, bicyclo[6.1.0]non-4-ene.

The invention relates more specifically to preferred spherical particles as defined above, characterised in that the monocyclic or polycyclic alkenes from which the monomer units are derived are:
norbornene (bicyclo[2.2.1]hept-2-ene) leading to a polymer comprising monomer units of formula (Z2c),
tetracyclododecadiene leading to a polymer comprising monomer units of formula (Z3c),
dicyclopentadiene leading to a polymer comprising monomer units of formula (Z3b),
the dimer of norbornadiene leading to a polymer comprising monomer units of formula (Z3a),
cycloocta-1,5-diene leading to a polymer comprising monomer units of formula (Z1i).

Advantageously the spherical particles as defined above are characterised in that at least 0.5% up to 100% of the monomer units are substituted by a chain R as defined above.

The invention relates more specifically to spherical particles as defined above, characterised in that they comprise:
between about 0.5% and 99.5% of monomer units substituted by a chain R as defined above, the said chain R being identical for these monomers,
and between about 0.5% and 99.5% of monomer units substituted by a chain R as defined above, the said chain R of these monomers being different from the chain R of the preceding monomers,
and/or between about 0.5% and 99.5% of monomer units directly substituted by a group X as defined above, this group X of these monomers being identical to or different from the group X of the chain R of the preceding monomers,
and/or between about 0.5% and 99.5% of unsubstituted monomer units,
   the total of the percentages of the different monomers mentioned above being 100%.

The invention relates more specifically to spherical particles as defined above, characterised in that the chain or chains R substituting the monomers are represented by the formula

—CH₂—O—(CH₂—CH₂—O)ₙ—CH₂—CH₂—O—X

wherein n is as defined above, and X represents H, —CH₂—COOH, —CH₂—COCl, —CH₂—COY, wherein Y depicts an active ingredient, or a biological molecule such as a protein.

The invention also relates to spherical particles as defined above, characterised in that the chain or chains comprise an ethylene polyoxide of formula (A) bonded covalently to the said monomer units by a hydrolysable bridge.

Such spherical particles are especially advantageous insofar as they permit a controlled release of the active ingredients which are stable or unstable in vivo. According to this strategy the release of the active ingredient trapped inside the particle, and therefore isolated from the external medium, and bonded covalently to the particle, is effected by a first step of destabilisation of the said particles by breaking the bonds between the monomer units and the chains R via an external stimulus (such as pH, hyperthermia . . . ), which involves salting out of the stabilising chains R. In a second reaction time the resulting chains R or Z1, which are or are not functionalised by the active ingredient, undergo hydrolysis reactions and release the active ingredient.

The spherical particles according to the invention are stimulable particles, that is to say they are sensitive to an external stimuli such as a variation in pH or in temperature, which then allows the release of the active ingredients trapped inside these particles.

Preferably, the hydrolysable bridges mentioned above are chosen from amongst the chain formations having approximately 1 to 10 units of ε-caprolactone, or functions selected from —OC(O)—, —C(O)OC(O)—, C(O)—NH— . . . .

In this connection, the invention relates more specifically to spherical particles as defined above, characterised in that the chain or chains R comprising an ethylene polyoxide of formula (A) bonded covalently to a hydrolysable bridge chosen from amongst the chain formations having approximately 1 to 10 units of ε-caprolactone are represented by the formula

—CH₂—(O—CO—(CH₂)₅)ₜ—O—CO—(CH₂)₅—O—CO—(CH₂)₂—CO—O—(CH₂—CH₂—O)ₙ—(CH₂)₂—O—X

wherein t represents an integer between 1 and 10, and X represents H, —CH₂—COOH, —CH₂—COCl or —CH₂—COY, Y representing an active ingredient, or a biological molecule such as a protein.

The invention also relates to spherical particles as defined above, characterised in that the active ingredient is chosen from molecules used in therapy, cosmetics, perfumery, or for surface coatings, such as paints and antifouling coatings.

The invention relates more specifically to spherical particles as defined above, characterised in that the active ingredient is a medicament used in therapy chosen in particular from amongst those in the following therapeutic categories: antiinflammatories, in particular indomethacin, anti-cancer treatments, antibiotics, anticoagulants or antimitotics.

The invention relates more specifically to spherical particles as defined above, characterised in that the biological molecule is chosen from amongst the proteins capable of bonding to an intracellular or extracellular biological target, or to antibodies or to any other specific ligand.

The invention relates more particularly to spherical particles as defined above, characterised in that the biological molecule is chosen from amongst the following proteins: avidine, albumin, growth factors such as VEGF.

The invention also relates to pharmaceutical compositions comprising spherical particles as defined above, wherein the different group or groups X contain a medicinally active ingredient, optionally in association with a pharmaceutically acceptable carrier, in particular for use in parenteral form.

The invention also relates to cosmetic compositions comprising spherical particles as defined above, in which the group(s) X contain an active ingredient used in cosmetics, optionally in association with a suitable carrier, especially for an application in the form of emulsions, creams.

The invention also relates to compositions for surface coatings comprising spherical particles as defined above, wherein the group(s) X contain an active ingredient used for the surface coatings, optionally in association with an appropriate carrier.

The invention also relates to a method of preparation of spherical particles as defined above, characterised in that it comprises a step of polymerisation of a monocyclic or polycyclic alkene as defined above substituted by a chain R as defined above, optionally in the presence of:
one or several monocyclic or polycyclic alkenes as defined above, identical to or different from the foregoing, and substituted by a chain R as defined above, the said chain R being different from that substituting the aforementioned monocyclic or polycyclic alkene,
and/or one or several monocyclic or polycyclic alkenes as defined above, identical to or different from the foregoing, and substituted by a group X as defined above, this group X being identical to or different from the group X of the chain R of the preceding alkenes,
and/or one or several monocyclic or polycyclic alkenes as defined above, identical to or different from the foregoing, the said alkenes being unsubstituted,
   the said polymerisation being carried out while stirring in the presence of a transition metal complex as initiator of the reaction chosen in particular from amongst those in groups IV or VI or VII, such as ruthenium, osmium, molybdenum, tungsten, iridium, titanium, in a polar or apolar medium, particularly with the aid of the following ruthenium-based complexes: RuCl₃, RuCl₂(PCy₃)₂CHPh.

The invention also relates to monocyclic or polycyclic alkenes, characterised in that they are substituted by a chain R or a group X as defined above, the alkene of the following formula being excluded:

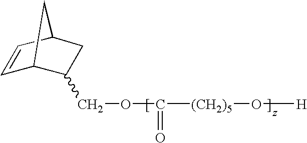

wherein z represents an integer between 1 and 340.

The preferred monocyclic or polycyclic alkenes as defined above are chosen from amongst those mentioned above.

The invention relates more particularly to monocyclic or polycyclic alkenes as defined above, characterised by the following formulae:

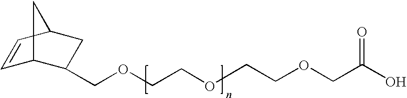

in which n represents an integer between approximately 50 and 340,

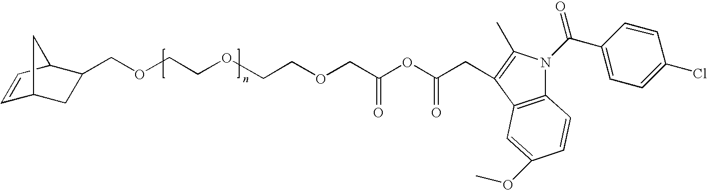

in which n represents an integer between approximately 50 and 340,

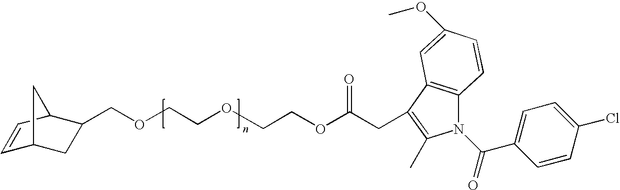

in which n represents an integer between approximately 50 and 340,

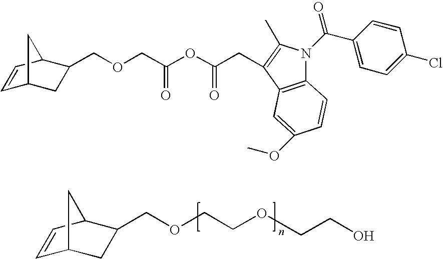

in which n represents an integer between approximately 50 and 340,

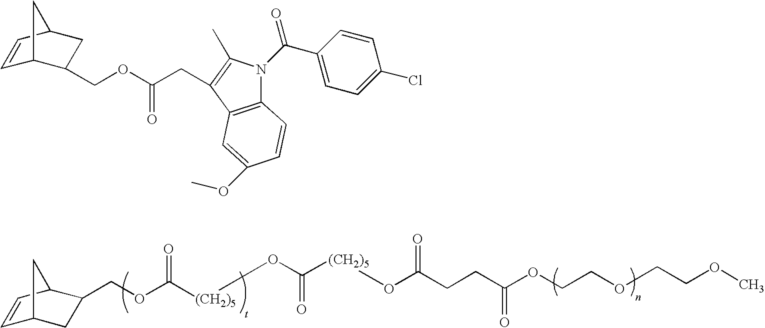

in which t represents an integer between 1 and 10 and n represents a whole number between approximately 50 and 340.

The invention also relates to the use of monocyclic or polycyclic alkenes as defined above for carrying out a method of preparation of spherical particles defined above, especially by the method described above.

The invention will now be illustrated in support with the following detailed description of obtaining spherical particles according to the invention and the physicochemical characteristics of the particles.

### A) Synthesis of Macromonomers of Formulae A and B Below

The macromonomers (A and B) are poly(ethylene oxide) oligomers with a molar mass ( M̅ₙ) of 7000 g/mol. They are derived from a “living” anionic polymerisation which allows control of the length and the functionality of the chains. They are functionalised at one of their ends by a norbornenyl unit, an entity chosen for its high reactivity in polymerisation by metathesis and, at the other end by a reactive function of the alcohol, acid, amine . . . type (A), or by the active ingredient (indomethacin) (B) via a cleavable bridge (acid anhydride, ester, amide, . . . ).

1. α-norbornenyl-ω-carboxylic acid-poly(ethylene Oxide); Formula A
Chemical formula:

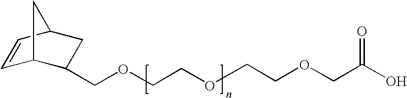

with n between 50 and 340 as a function of the requirements of the envisaged application.

### Reference: NB-POE-COOH.

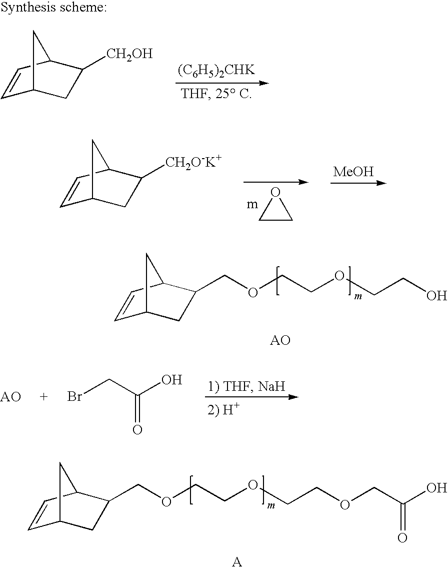

### Procedure for Synthesis:

5-norbornene-2-methanol (0.5 mL) in solution in tetrahydrofuran (THF) (200 mL) is first of all deprotonated by the addition of a molar equivalent of diphenylmethyl potassium. The resulting radical will then initiate the polymerisation of ethylene oxide (28 mL) in a “live” manner (48 h) until the destruction of the active centres by the addition of methanol (1 mL). The alcohol function of the poly(ethylene oxide) obtained (A0) will then be transformed into an acid function by deprotonation of A0 (10 g) with NaH (0.17 g) in solution in THF (15 mL), followed by the addition of bromoacetic acid (0.42 g). After washing of the product with hydrochloric acid (18 mL, 1M) then precipitation in ether, the macromonomer A is obtained in a pure form.

2. α-norbornenyl-ω-indomethacin-poly(ethylene Oxide); Formula B
Chemical formula:

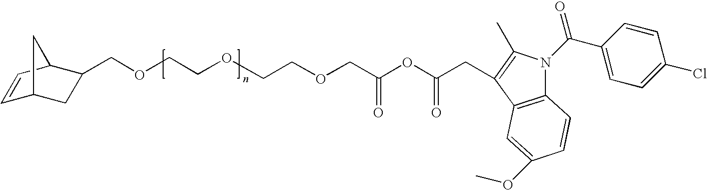

where n is between 70 and 200 as a function of the requirements of the envisaged application.

### Reference: NB-POE-CO(O)-IND.

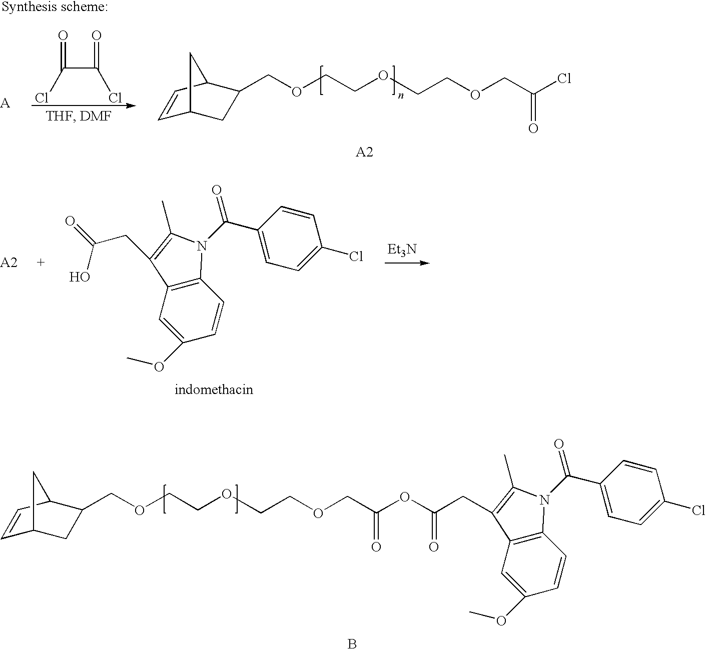

### Procedure for Synthesis:

The acid function of NB-POE-COOH (A) is transformed into acid chloride (A2) by reaction of A (5.2 g) on oxalyl chloride (0.08 mL) in THF (25 mL) in the presence of a catalytic quantity of dimethylformamide for 24 h. Indomethacin (0.6 g) as well as triethylamine (0.24 mL) are then added to the solution of A2 and left while stirring for 15 h. After precipitation in ether, the macromonomer B is obtained.

3. Indomethacin Derivative of norbornene

The monomer used in the preceding reactions is norbornene (NBH) or norbornene functionalised (NBD) by the active ingredient. This latter is then introduced via a hydrolysable bridge of the type of ester, anhydride, amide . . . . The synthesis of norbornene functionalised by indomethacin is described below.

### Chemical Formula:

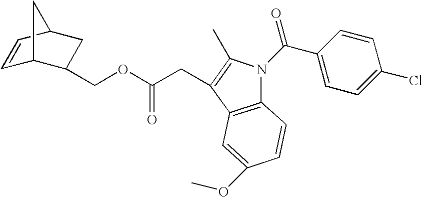

### Reference: NBD.

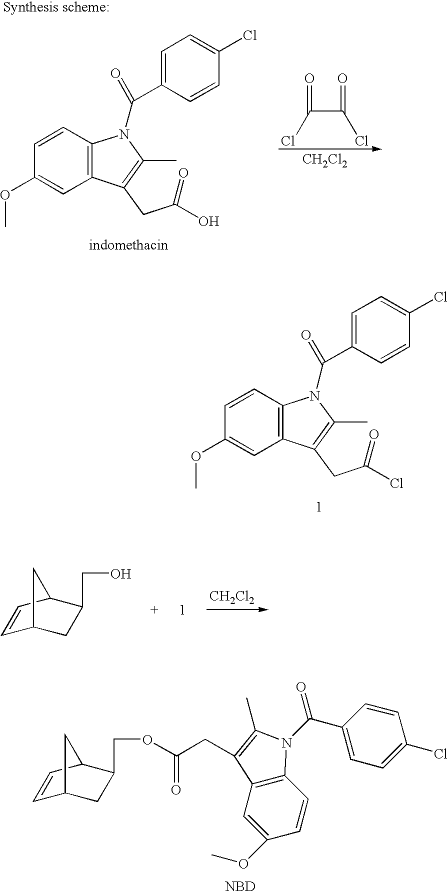

### Procedure for Synthesis:

### Synthesis of the Monomer NBD

During a typical reaction, oxalyl chloride (0.87 mL) is added to indomethacin (1.1 g) in solution in dichloromethane (20 mL). After 2 hours of reaction and elimination of the unreacted oxalyl chloride, the compound 1 obtained is then added to a solution of 5-norbornene-2-methanol (0.36 mL) in dichloromethane (20 mL) in the presence of triethylamine (0.84 mL) and left while stirring for 15 hours at 45° C. After purification by extraction, the monomer NBD is obtained (ρ>95%).

### B. Synthesis of Particles

The particles according to the invention are obtained by copolymerisation in a dispersed medium (emulsion, mini-emulsion and micro-emulsion, dispersion, suspension) of vinyl monomers (cyclo-olefins) with macromonomers α,ω-functionalised by a polymerisable entity and a reactive function or an active ingredient (medicaments, organic molecules . . . ). The polymerisation is initiated by transition metals and can be carried out in an aqueous or organic medium (dichloromethane/ethanol). Macromonomers play the part of stabiliser and functionalising agent. In the capacity of stabilisers they make it possible during the formation of the polymer in the reaction medium to disperse it in the form of spherical nanoparticles. From the purely steric point of view the stabilisation is insensitive to any variation in pH of the medium. Moreover, the functionalisation of latex by means of a macromonomer improves the availability of the reactive functions on the surface of the latex and preserves the reactivity thereof.

The initiator of the polymerisation is a ruthenium-based complex which is stable in a polar medium: RuCl₃, RuCl₂(PCy₃)₂CHPh and homologues thereof. Latex synthesised in these conditions will consist of polyalkenamer chains bearing poly(ethylene oxide) grafts which will serve to stabilise the particles.

The particles obtained are stable in an aqueous and/or organic medium. Their size is between a few nanometres and a few micrometres as a function of the method of polymerisation used (dispersion, suspension, mini-emulsion . . . ). The nanoparticles are spherical with very good isometry.

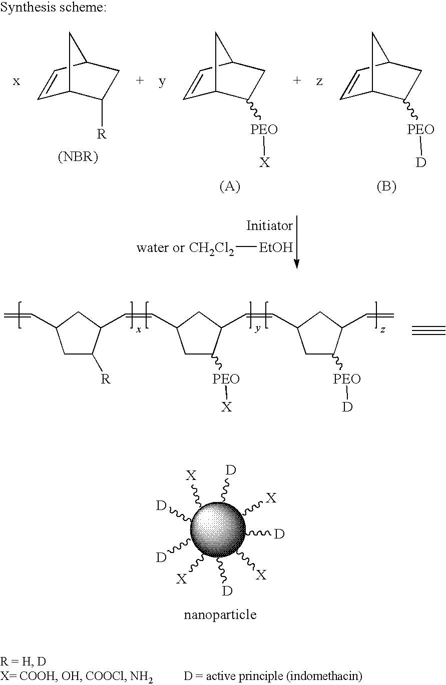

### Procedure for Synthesis:

The macromonomers A and B are copolymerised in the presence of a monomer (NBH and/or NBD). In a typical reaction 0.8 g of monomer and 1 g of macromonomer (0.2 g of A and 0.8 g of B) previously dissolved in 14 ml of a dichloromethane/ethanol mixture (35%/65%) are added under a nitrogen atmosphere and with vigorous stirring to 10 ml of dichloromethane/ethanol (50%/50%) containing 20 mg of initiator. The duration of the polymerisation is one hour. The totally homogeneous starting medium becomes increasingly cloudy as the polymerisation takes place. Monitoring of the polymerisations by gas chromatography has revealed total conversions of monomers in less than one minute. The incorporation of the macromonomers A and B into the latex is total.

### C. Variant for the Transport of Sensitive Active Ingredient

The latex is prepared as previously by copolymerisation between a cycloolefin (norbornene) which does or does not carry an active ingredient (indomethacin) and the stabilising polymer (NB-PCL-POE-OMe). This latter, which is or is not functionalised by a reactive function of the acid, acid chloride, alcohol, amine type (same function as previously), has a hydrolysable bridge, particularly units of ε-caprolactone (PCL) between the polymerisable function and the ethylene polyoxide chain according to the following scheme:

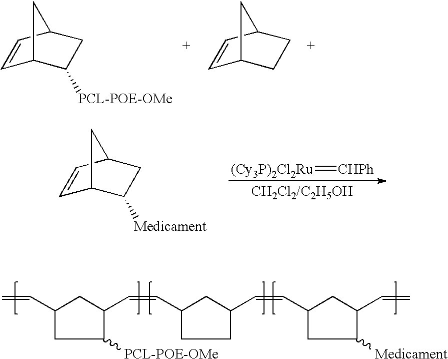

According to this process the release of the active ingredient trapped inside the particle and bonded covalently thereto (FIG. 13) necessitates a first step of destabilisation of the latex. This can be achieved via an external stimulus (pH, hyperthermia . . . ) by salting out of the stabilising chains.

In a second reaction time the resulting linear chains of polyalkenamers functionalised by the active ingredient undergo hydrolysis reactions and release the active ingredient (FIG. 14).

1) Procedure for synthesis of the copolymer poly(caprolactone-β-ethylene glycol)-α-norbornene-ω-methyl ether NB-PCL-POE-OMe.

Preparation of poly(caprolactone)α-norbornenyl (NB-Pcapro)

Triethyl aluminium (1.3×10⁻² moles) is added drop by drop to a solution of 2-hydroxymethyl-5-norbornene (1.3×10⁻² moles) in toluene (100 mL) cooled to −80° C. After a progressive return to ambient temperature the reaction is continued for 2.5 hours. Caprolactone (3.9 mole) is then added to the reaction medium with vigorous stirring. After 18 hours of reaction, 50 mL of hydrochloric acid (0.1 N) are added. After washing until neutral poly(ε-caprolactone)α-norbornenyl is precipitated cold in heptane then filtered on frit No. 4. The traces of heptane will be eliminated by heating (40° C.) in vacuo for 10 hours. The polymer obtained is then freeze-dried three times with dioxan as solvent.

Preparation of poly(ethylene Glycol)-α-carboxylic Acid-ω-methyl Ether

Solubilise 3.89×10⁻³ moles of succinic anhydride and 4.10×10⁻³ moles of triethylamine in 45 mL of anhydrous acetone. Whilst stirring, add drop by drop a solution of poly(ethylene glycol) monomethyl ether (6×10⁻⁴ moles) in 15 mL of anhydrous CH₂Cl₂. After 16 hours of reaction, add 1 mL of methanol. After concentration in a rotary evaporator, precipitate the polymer in ethyl ether. Recommence the steps of dissolution/precipitation two further times. Place the polymer in a dynamic vacuum for 10 hours to eliminate all traces of solvent.

Preparation of the Copolymer poly(caprolactone-b-ethylene Glycol)-α-norbornene-ω-methyl Ether (NB-Pcapro-PEG-OMe)

Solubilise 4×10⁻⁴ moles of poly(ethylene glycol)-a-carboxylic acid-ω-methyl ether in 40 mL of anhydrous CH₂Cl₂. Add oxalyl chloride (8×10⁻⁴ moles) to this solution cooled to 5° C. After 15 hours of reaction, remove the excess of unreacted oxalyl chloride as well as the CH₂Cl₂ under reduced pressure. The yellow residue obtained is then redissolved in 40 mL of dichloromethane. After having added triethylamine (4.3×10⁻⁴ moles), add α-norbornenyl poly(caprolactone). After concentration in a rotary evaporator, precipitate the polymer in ethyl ether. Recommence the steps of dissolution/precipitation two further times. Place the polymer under reduced pressure for 10 hours to eliminate all traces of solvent.

The synthesis of a-norbornenyl poly(caprolactone) is effected according to the following scheme:

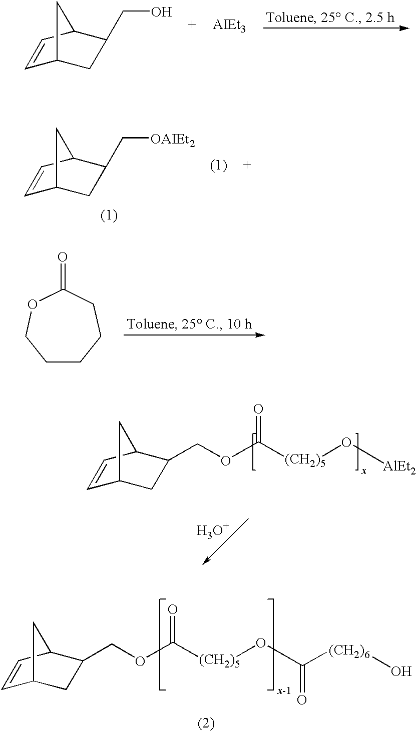

The synthesis of poly(ε-caprolactone-b-ethylene glycol)-a-norbornene-ω-methyl ether is effected according to the following scheme:

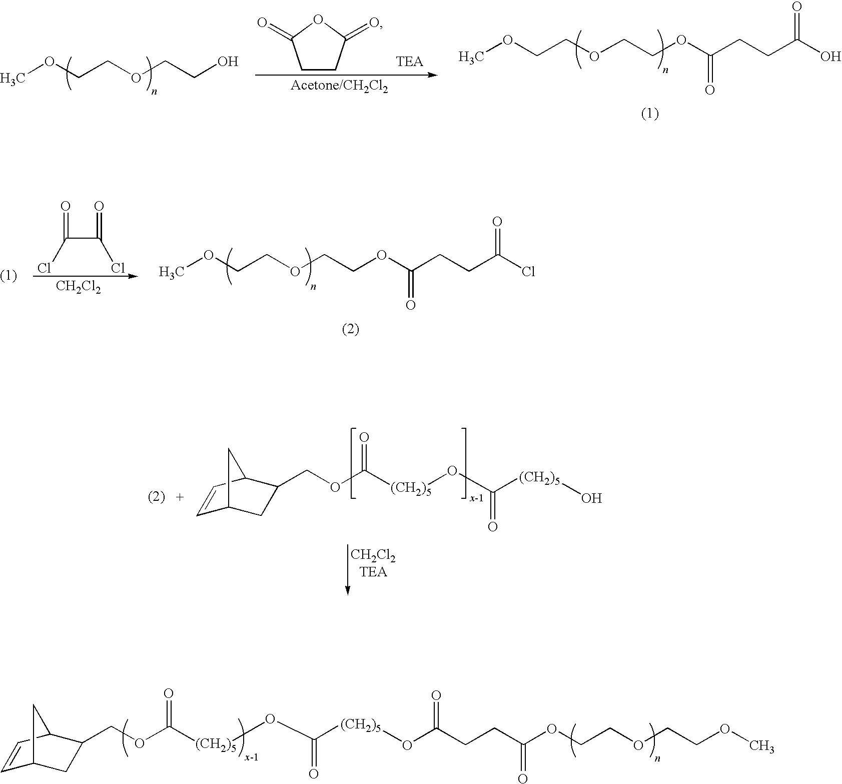

### 2) Release of the Active Ingredient

Once the particle was synthesised we verified by UV-visible spectrometry the possibility of releasing the medicament by simple lowering of the pH. The results obtained allowed confirmation of a progressive and controlled release of indomethacin. Moreover, the application of a pH equal to 3 revealed that more than 85% thereof could be salted out in 48 hours.

### LEGENDS ON THE DRAWINGS

FIG. 1: RMN ¹H NMR spectrum of the macromonomer of formula A.

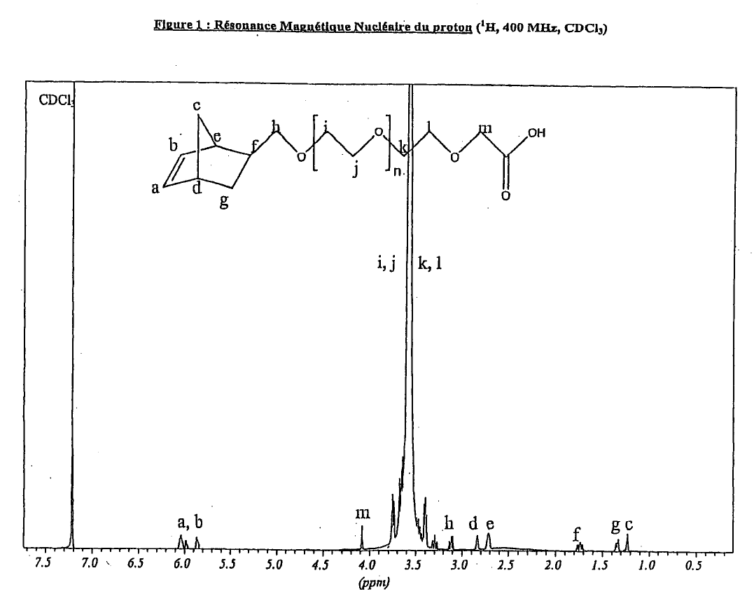

FIG. 2: RMN ¹³C NMR spectrum of the macromonomer of formula A.

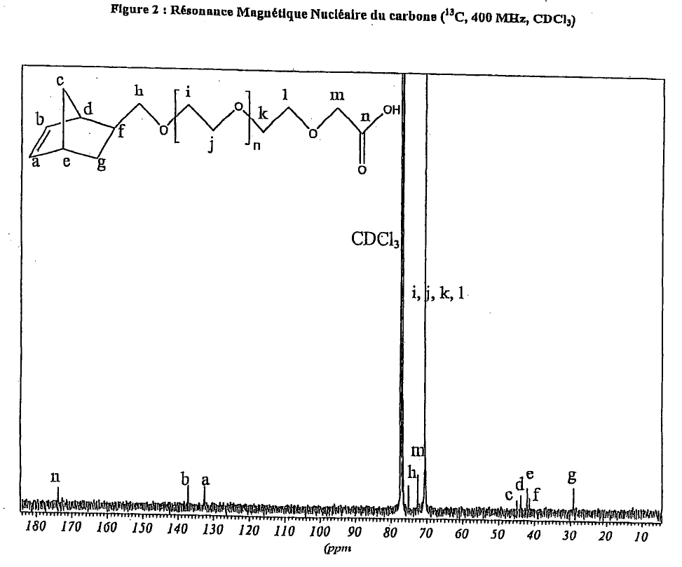

FIG. 3: Steric exclusion chromatography of the macromonomer of formula A in THF.

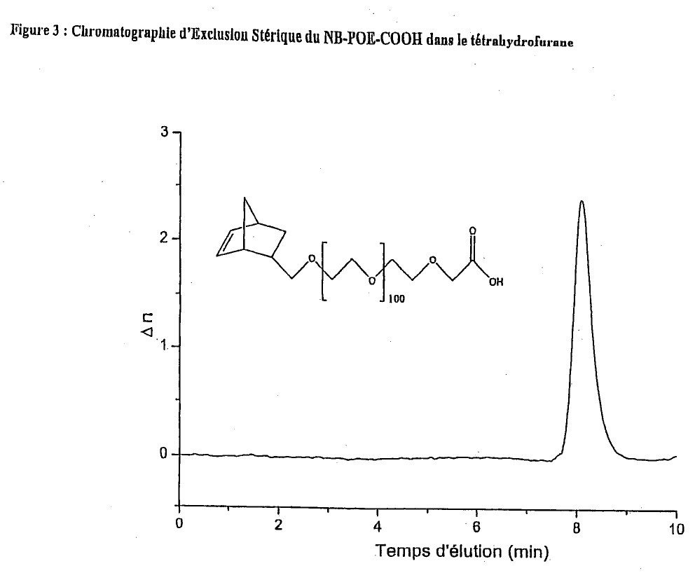

FIG. 4: RMN ¹H NMR spectrum of the macromonomer of formula B.

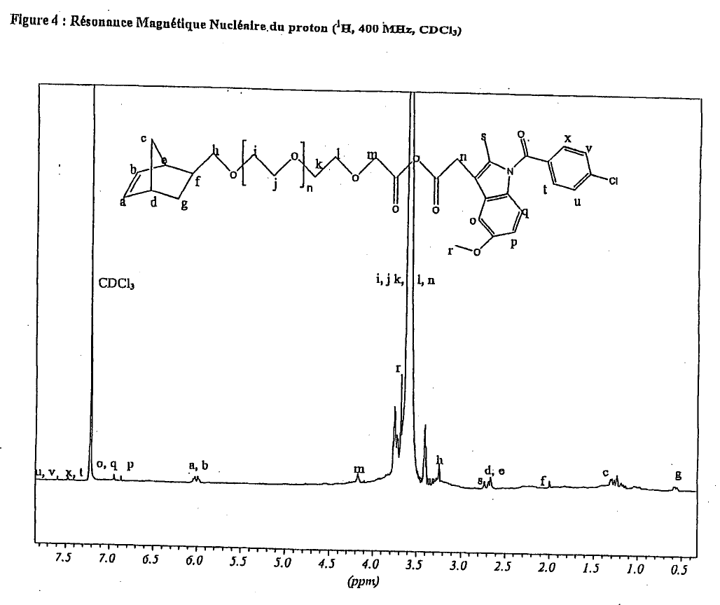

FIG. 5: Steric exclusion chromatography of the macromonomer of formula B in THF.

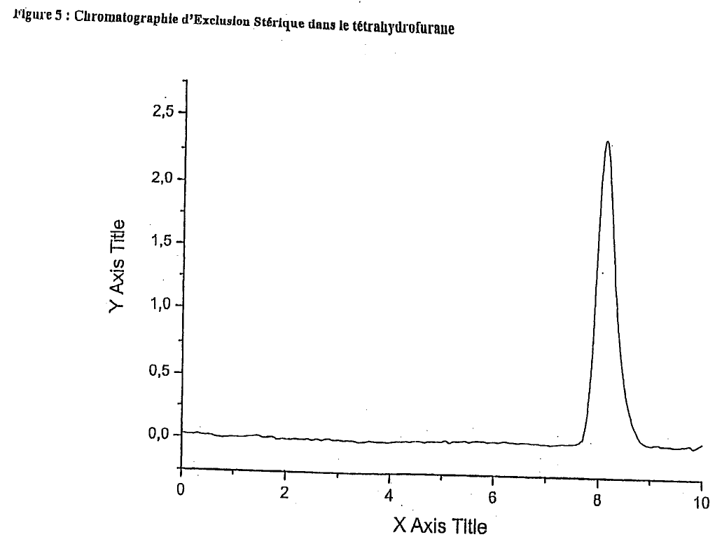

FIG. 6: RMN ¹H NMR spectrum of the compound NBD.

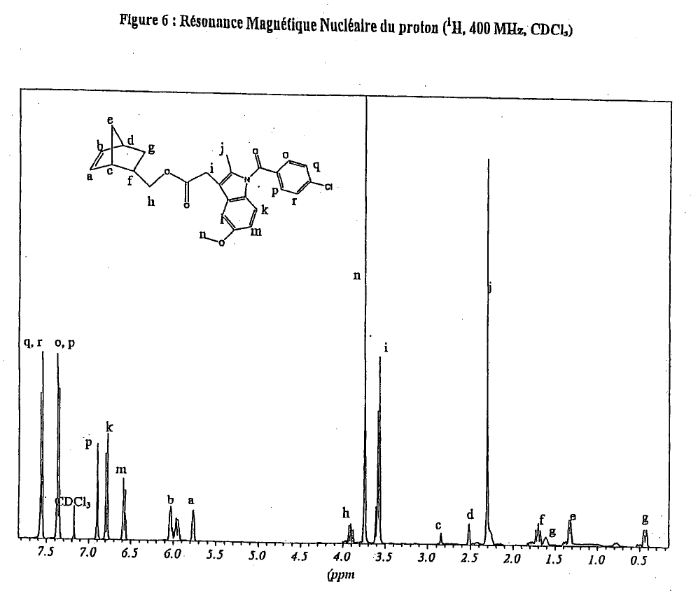

FIG. 7: RMN ¹³C NMR spectrum of the compound NBD.

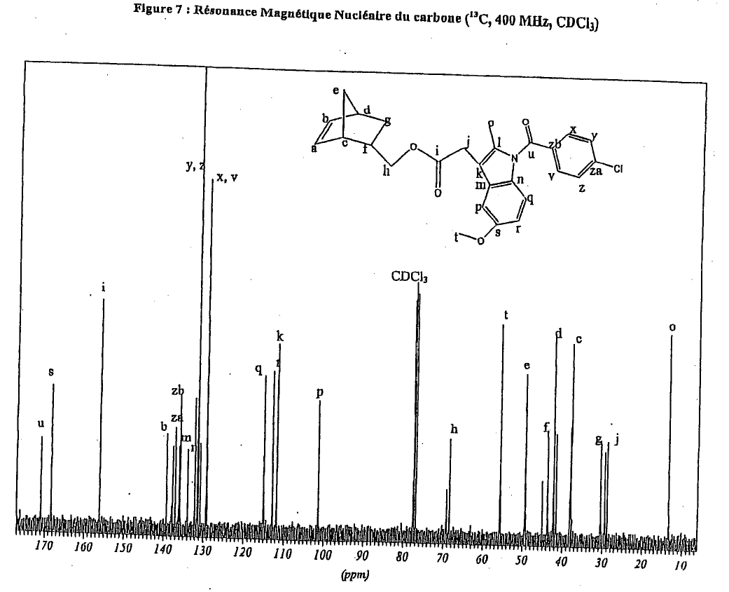

FIG. 8: Study of the conversion to NB and to NB-POE-CO(O)—IND during the polymerisation reaction. Evolution of the conversion to norbornene (♦, NB) and of the macromonomer (, NB-POE-CO(O)—IND) as a function of time.

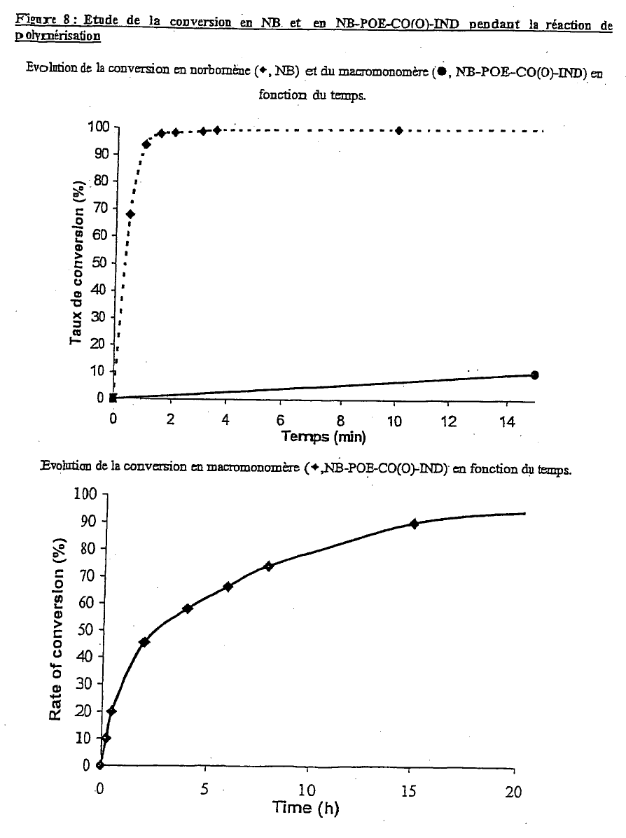

FIG. 9: Scanning electron microscope image of spherical particles obtained by copolymerisation of the macromonomers A and B in the presence of the monomers NBH and/or NBD.

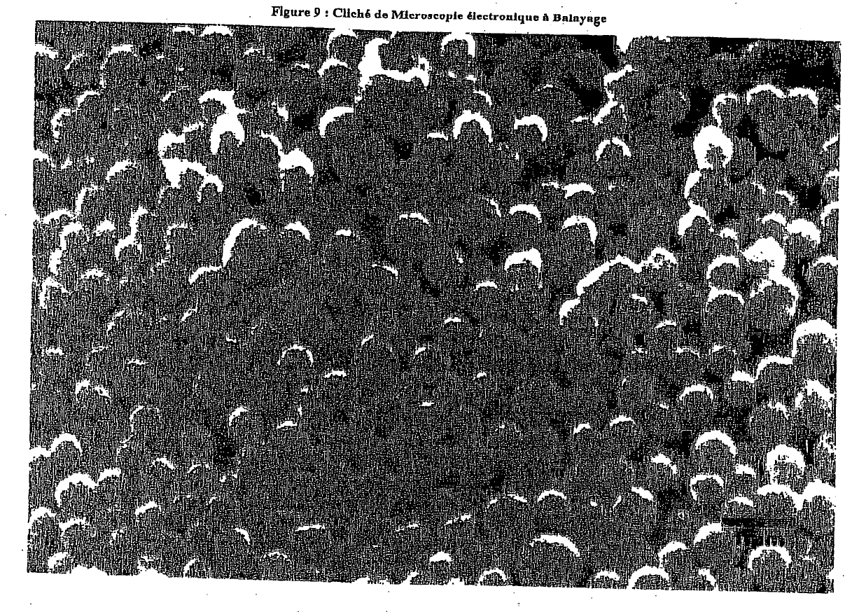

FIG. 10: Transmission electron microscope image of spherical particles obtained by copolymerisation of the macromonomers A and B in the presence of the monomers NBH and/or NBD.

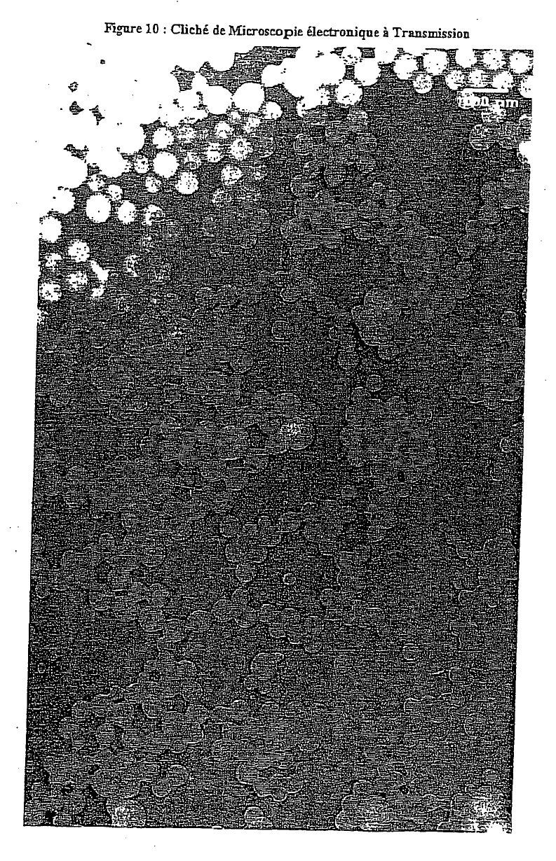

FIG. 11: Size and size distribution of the spherical particles obtained by copolymerisation of the macromonomers A and B in the presence of the monomers NBH and/or NBD, by dynamic diffusion of light.

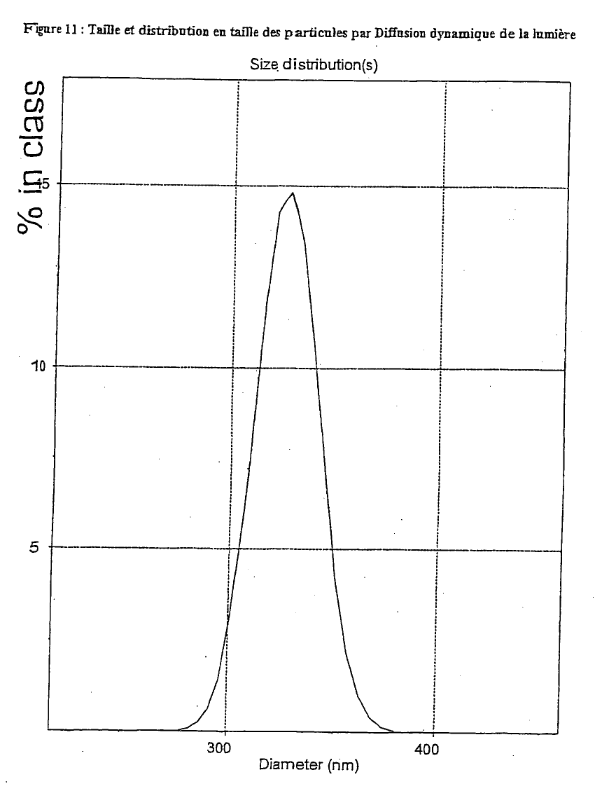

FIG. 12: Steric exclusion chromatography of the spherical particles obtained by copolymerisation of the macromonomers A and B in the presence of the monomers NBH and/or NBD in THF.

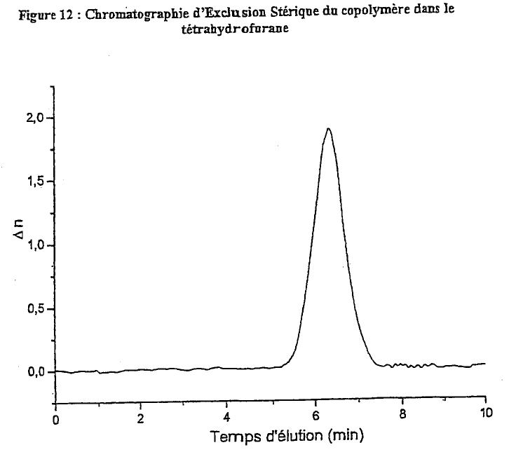

FIG. 13: Representation of a spherical particle according to the invention in which the active ingredient is trapped inside the particle and bonded covalently thereto.

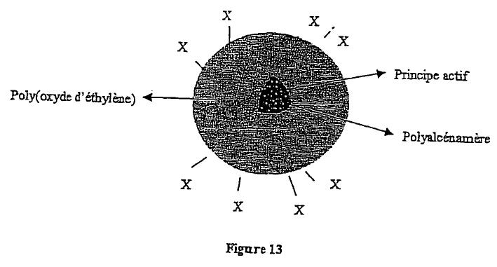

FIG. 14: Illustration of the destabilisation of a spherical particle according to the invention (or latex) and salting out of the medicament.

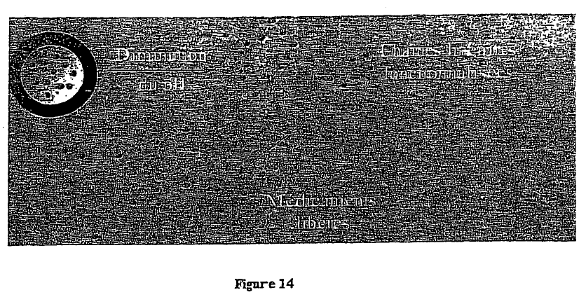

1-23. (canceled)

24. Spherical particles having a diameter between 10 nm and 100 μm, said particles being formed by polymer chains containing about 30 to 10000 monomer units, identical or different, derived from the polymerisation of monocyclic alkenes wherein the number of carbon atoms constituting the ring is of about 4 to 12 or polycyclic alkenes, wherein the total number of carbon atoms constituting the rings is of about 6 to 20, and wherein at least one of the said monomer units are substituted by a chain R comprising an ethylene polyoxide of formula (A) which are covalently bonded to the said monomer units via a hydrolysable bridge or not covalently bonded via such a hydrolysable bridge
—(CH₂—CH₂—O)ₙ—X (A)
wherein n represents an integer from about 50 to 340, especially from 70 to 200, and X represents an alkyl or alkoxy chain with about 1 to 10 carbon atoms, comprising a reactive function of the OH, halogen, NH₂, C(O)X₁ type in which X₁ represents a hydrogen atom, a halogen atom, an OR′ or NHR′ group wherein R′ represents a hydrogen atom or a hydrocarbon chain with approximately 1 to 10 carbon atoms, substituted or unsubstituted, where X represents a polyene or another group comprising a photosensitive function, the said reactive function being optionally engaged in a bond with an active ingredient or a protein or another biological molecule, the said chain R being bonded covalently to the said monomers.

25. The spherical particles of claim 24, wherein the monomer units are derived from the polymerisation of monocyclic alkenes and are of the following formula (Z1)
═[CH—R₁—CH]═ (Z1)
wherein R₁ represents a hydrocarbon chain with 2 to 10 carbon atoms, saturated or unsaturated, the said monomers being optionally substituted by a chain R, or directly by a group X, as defined in claim 1.

26. The spherical particles of claim 24, wherein the monocyclic alkenes from which the monomer units are derived are:
cyclobutene leading to a polymer comprising monomer units of formula (Z1a) below: 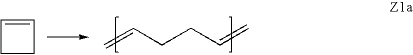
cyclopentene leading to a polymer comprising monomer units of formula (Z1b) below: 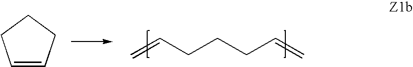
cyclopentadiene leading to a polymer comprising monomer units of formula (Z1c) below: 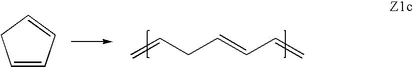
cyclohexene leading to a polymer comprising monomer units of formula (Z1d) below: 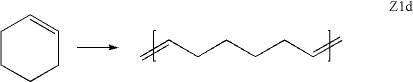
cyclohexadiene leading to a polymer comprising monomer units of formula (Z1e) below: 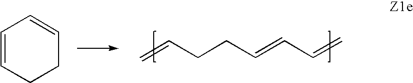
cycloheptene leading to a polymer comprising monomer units of formula (Z1f) below: 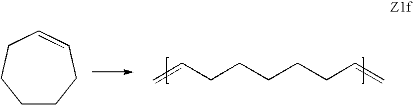
cyclooctene leading to a polymer comprising monomer units of formula (Z1h) below: 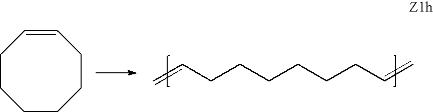
cyclooctapolyene, especially cycloocta-1,5-diene, leading to a polymer comprising monomer units of formula (Z1i) below: 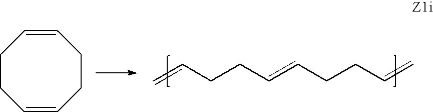
cyclononene leading to a polymer comprising monomer units of formula (Z1j) below: 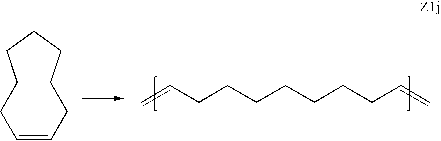
cyclononadiene leading to a polymer comprising monomer units of formula (Z1k) below: 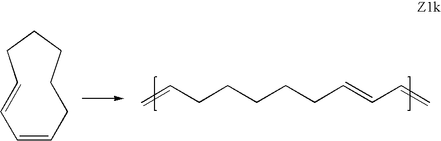
cyclodecene leading to a polymer comprising monomer units of formula (Z1l) below: 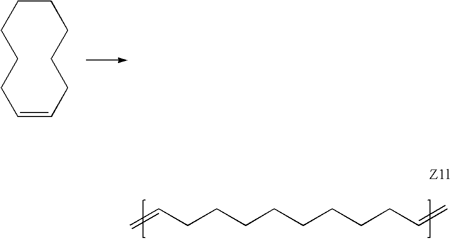
cyclodeca-1,5-diene leading to a polymer comprising monomer units of formula (Z1m) below: 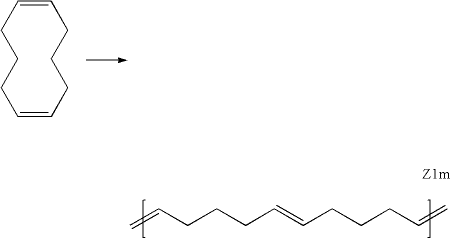
cyclododecene leading to a polymer comprising monomer units of formula (Z1n) below: 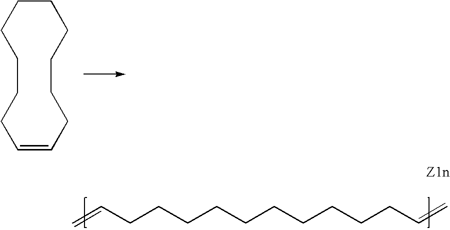
or also 2,3,4,5-tetrahydrooxepin-2-yl acetate, cyclopentadecene, paracyclophane, ferrocenophane.

27. The spherical particles of claim 24, wherein the monomer units are derived from the polymerisation of polycyclic alkenes and are:
of formula (Z2) below:
═[CH—R₂—CH]═ (Z2)
in which R₂ represents
a ring of formula 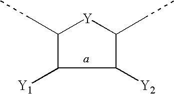
in which:
Y represents —CH₂—, or a heteroatom, or a —CHR— group, or a —CHX— group, R and X being as defined above,
Y₁ and Y₂ independently of one another represent H, or a chain R, or a group X, as mentioned above, or form in association with the carbon atoms bearing them a ring with 4 to 8 carbon atoms, this ring being optionally substituted by a chain R or a group X as mentioned above,
a represents a single or double bond,
or a ring of formula 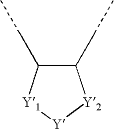
in which:
Y′ represents —CH₂—, or a heteroatom, or a —CHR— group, or a —CHX— group, R and X being as defined above,
Y′₁ and Y′₂ independently of one another represent —CH₂—, or a —C(O) group, of a —COR group, or a —C—OX group, R and X being as defined above,
of formula (Z3) below: 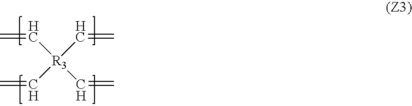
in which R₃ represents:
a ring of formula 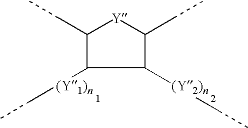
in which:
n₁ and n₂, independently of one another, represent 0 or 1,
Y″ represents —CH₂—, or a —CHR— group, or a —CHX— group, R and X being as defined above,
Y″₁ and Y″₂ independently of one another represent a hydrocarbon chain with 0 to 10 carbon atoms,
or a ring of formula 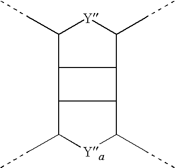
in which Y″ and Y″a independently of one another represent —CH₂—, or a —CHR— group, or a —CHX— group, R and X being as defined above,
or a ring of formula 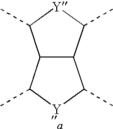
in which Y″ and Y″a independently of one another represent —CH₂—, or a —CHR— group, or a —CHX— group, R and X being as defined above.

28. The spherical particles of claim 24, wherein the polycyclic alkenes from which the monomer units are derived are:
monomers containing a cyclobutene ring leading to a polymer comprising monomer units of formula (Z2a) below: 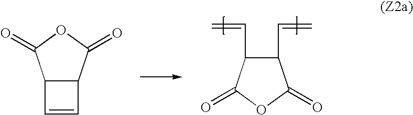
monomers containing a cyclopentene ring leading to a polymer comprising monomer units of formula (Z2b) below: 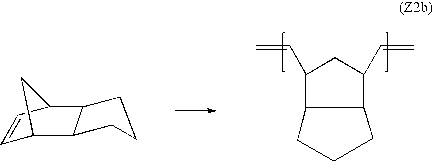
(bicyclo[2.2.1]hept-2-ene)norbornene leading to a polymer comprising monomer units of formula (Z2c) below: 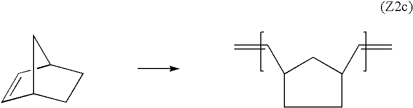
norbornadiene leading to a polymer comprising monomer units of formula (Z2d) below: 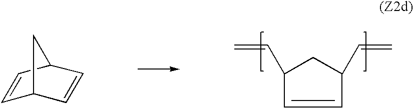
7-oxanorbornene leading to a polymer comprising monomer units of formula (Z2e) below: 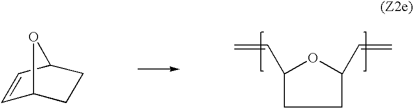
7-oxanorbornadiene leading to a polymer comprising monomer units of formula (Z2f) below: 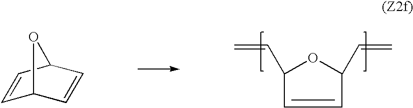
the dimer of norbornadiene leading to a polymer comprising monomer units of formula (Z3a) below: 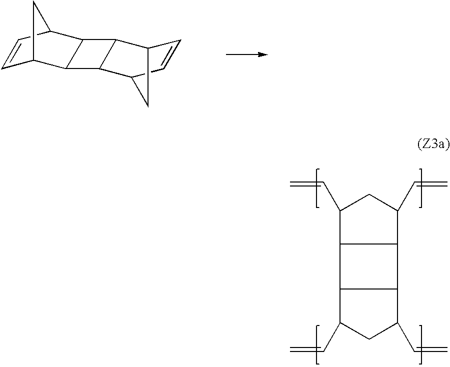
dicyclopentadiene leading to a polymer comprising monomer units of formula (Z3b) below: 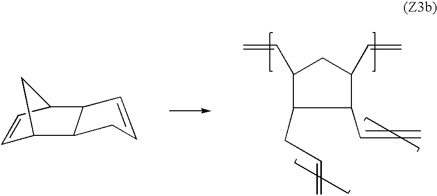
tetracyclododecadiene leading to a polymer comprising monomer units of formula (Z3c) below: 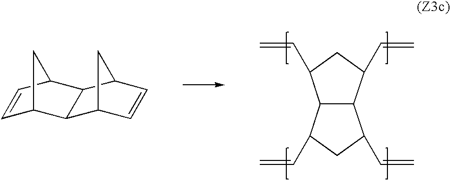
or bicyclo[5.1.0]oct-2-ene, bicyclo[6.1.0]non-4-ene.

29. The spherical particles of claim 24, wherein the monocyclic or polycyclic alkenes from which the monomer units are derived are:
norbornene (bicyclo[2.2.1]hept-2-ene) leading to a polymer comprising monomer units of formula (Z2c),
tetracyclododecadiene leading to a polymer comprising monomer units of formula (Z3c),
dicyclopentadiene leading to a polymer comprising monomer units of formula (Z3b),
the dimer of norbornadiene leading to a polymer comprising monomer units of formula (Z3a),
cycloocta-1,5-diene leading to a polymer comprising monomer units of formula (Z1i).

30. The spherical particles of claim 24, wherein at least 0.5% up to 100% of the monomer units are substituted by a R chain.

31. The spherical particles of claim 24, wherein said particles comprise:
between about 0.5% and to 99.5% of monomer units substituted by a chain R as defined above, said chain R being identical for these monomers,
and between about 0.5% and 99.5% of monomer units substituted by a chain R as defined above, the said chain R of these monomers being different from the chain R of the preceding monomers,
and/or between about 0.5% and 99.5% of monomer units directly substituted by a group X as defined above, this group X of these monomers being identical to or different from the group X of the chain R of the preceding monomers,
and/or between about 1% and 99.5% of unsubstituted monomer units,
the total of the percentages of the different monomers mentioned above being 100%.

32. The spherical particles of claim 24, wherein the chain or chains R substituting the monomers are represented by the formula
—CH₂—O—(CH₂—CH₂—O)ₙ—CH₂—CH₂—O—X
in which n is as defined above, and X represents H, —CH₂—COOH, —CH₂—COCl, —CH₂—COY, wherein Y depicts an active ingredient, or a biological molecule such as a protein.

33. The spherical particles according to claim 24, wherein the chain or chains comprise an ethylene polyoxide of formula (A) bonded covalently to the said monomer units by a hydrolysable bridge chosen from amongst the chain formations having approximately 1 to 10 units of ε-caprolactone, or —OC(O)—, —C(O)OC(O)—, C(O)—NH— functions.

34. The spherical particles of claim 24, wherein the chain or chains R comprising an ethylene polyoxide of formula (A) bonded covalently to a hydrolysable bridge chosen from amongst the chain formations having approximately 1 to 10 units of ε-caprolactone are represented by the formula
—CH₂—(O—CO—(CH₂)₅)ₜ—O—CO—(CH₂)₅—O—CO—(CH₂)₂—CO—O—(CH₂—CH₂—O)ₙ—(CH₂)₂—O—X
in which t represents an integer between 1 and 10, and X represents H, —CH₂—COOH, —CH₂—COCl or —CH₂—COY, Y representing an active ingredient, or a biological molecule such as a protein.

35. The spherical particles of claim 24, wherein the active ingredient is chosen from the molecules used in therapy, cosmetics, perfumery, or for surface coatings, such as paints and antifouling coatings.

36. The spherical particles of claim 24, wherein the active ingredient is a medicament used in therapy chosen especially from amongst those in the following therapeutic categories: antiinflammatories, in particular indomethacin, anti-cancer treatments, antibiotics, anticoagulants or antimitotics.

37. The spherical particles of claim 24, wherein the biological molecule is chosen from amongst the proteins capable of bonding to an intracellular or extracellular biological target, or to antibodies or to any other specific ligand.

38. The spherical particles of claim 37, wherein the biological molecule is chosen from amongst the following proteins: avidine, albumin, growth factors such as VEGF.

39. Pharmaceutical compositions comprising spherical particles according to claim 24, wherein the group(s) X contain a medicinally active ingredient, optionally in association with a pharmaceutically acceptable carrier, especially for use in parenteral form.

40. Cosmetic compositions comprising spherical particles according to claim 24, wherein the group(s) X contain an active ingredient used in cosmetics, optionally in association with a suitable carrier, especially for an application in the form of emulsions, creams.

41. Compositions for surface coatings comprising spherical particles according to claim 24, wherein the group(s) X contain an active ingredient used for the surface coatings, optionally in association with a suitable carrier.

42. Method of preparation of spherical particles as defined in claim 24, which comprises a step of polymerisation of a monocyclic or polycyclic alkene substituted by a chain R, this polymerisation step optionally being effected in the presence of:
one or several monocyclic or polycyclic alkenes, and substituted by a chain R, different from that substituting the aforementioned monocyclic or polycyclic alkene,
and/or one or several monocyclic or polycyclic alkenes, and substituted by a group X identical to or different from the group X of the chain R of the preceding alkenes,
and/or one or several monocyclic or polycyclic alkenes, identical to or different from the foregoing, said alkenes being unsubstituted,
the said polymerisation being carried out while stirring in the presence of a transition metal complex as initiator of the reaction chosen in particular from amongst those in groups IV or VI or VII, such as ruthenium, osmium, molybdenum, tungsten, iridium, titanium, in a polar or apolar medium, particularly with the aid of the following ruthenium-based complexes: RuCl₃, RuCl₂ (PCy₃)₂CHPh.

43. Monocyclic or polycyclic alkenes, which are substituted by a chain R or a group X as defined in claim 24, with the proviso that the alkene of the following formula is excluded: 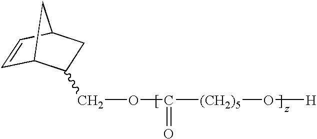
wherein z represents an integer between 1 and 340.

44. The monocyclic or polycyclic alkenes of claim 43, wherein the monocyclic alkenes are chosen from amongst:
cyclobutene leading to a polymer comprising monomer units of formula (Z1a) below: 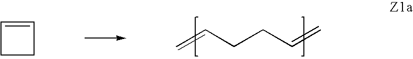
cyclopentene leading to a polymer comprising monomer units of formula (Z1b) below: 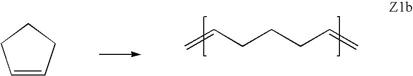
cyclopentadiene leading to a polymer comprising monomer units of formula (Z1c) below: 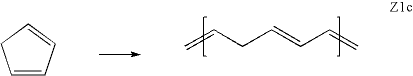
cyclohexene leading to a polymer comprising monomer units of formula (Z1d) below: 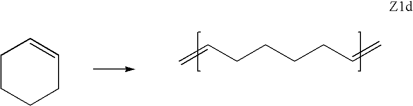
cyclohexadiene leading to a polymer comprising monomer units of formula (Z1e) below: 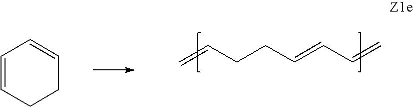
cycloheptene leading to a polymer comprising monomer units of formula (Z1f) below: 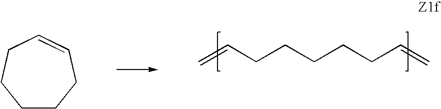
cyclooctene leading to a polymer comprising monomer units of formula (Z1h) below: 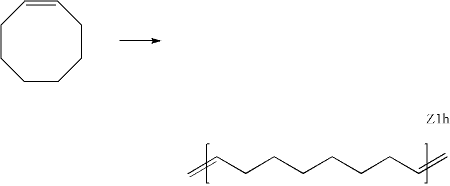
cyclooctapolyene, especially cycloocta-1,5-diene, leading to a polymer comprising monomer units of formula (Z1i) below: 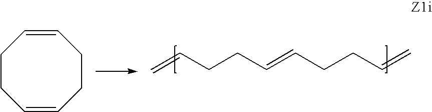
cyclononene leading to a polymer comprising monomer units of formula (Z1j) below: 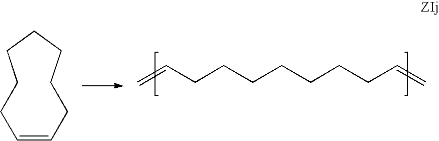
cyclononadiene leading to a polymer comprising monomer units of formula (Z1k) below: 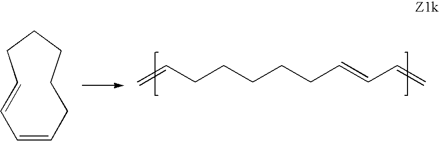
cyclodecene leading to a polymer comprising monomer units of formula (Z1l) below: 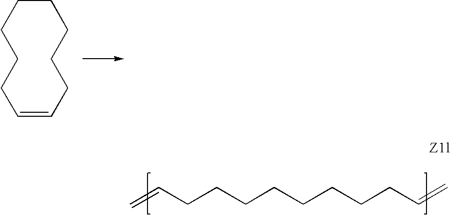
cyclodeca-1,5-diene leading to a polymer comprising monomer units of formula (Z1m) below: 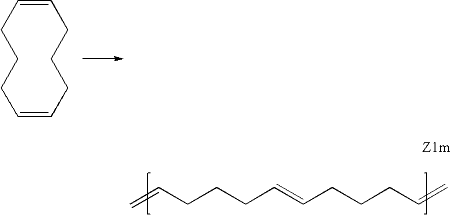
cyclododecene leading to a polymer comprising monomer units of formula (Z1n) below: 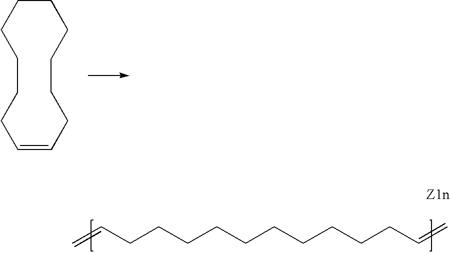
or also 2,3,4,5-tetrahydrooxepin-2-yl acetate, cyclopentadecene, paracyclophane, ferrocenophane; and the polycyclic alkenes are chosen from amongst:
monomers containing a cyclobutene ring leading to a polymer comprising monomer units of formula (Z2a) below: 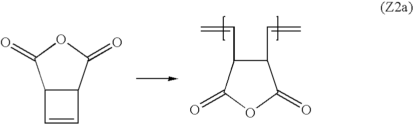
monomers containing a cyclopentene ring leading to a polymer comprising monomer units of formula (Z2b) below: 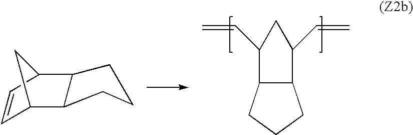
(bicyclo[2.2.1]hept-2-ene)norbornene leading to a polymer comprising monomer units of formula (Z2c) below: 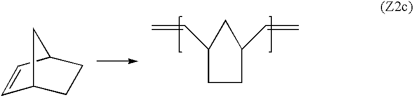
norbornadiene leading to a polymer comprising monomer units of formula (Z2d) below: 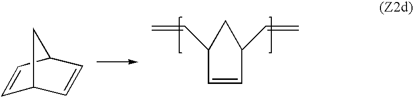
7-oxanorbornene leading to a polymer comprising monomer units of formula (Z2e) below: 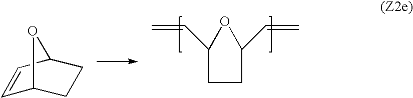
7-oxanorbornadiene leading to a polymer comprising monomer units of formula (Z2f) below: 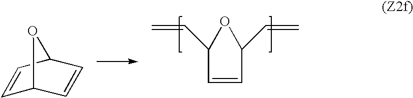
the dimer of norbornadiene leading to a polymer comprising monomer units of formula (Z3a) below: 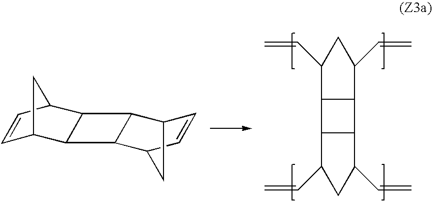
dicyclopentadiene leading to a polymer comprising monomer units of formula (Z3b) below: 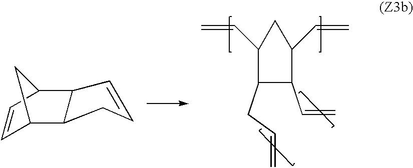
tetracyclododecadiene leading to a polymer comprising monomer units of formula (Z3c) below: 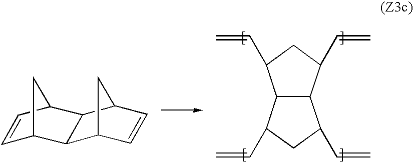
or bicyclo[5.1.0]oct-2-ene, bicyclo[6.1.0]non-4-ene.

45. The monocyclic or polycyclic alkenes of claim 43, which have one of the following formulae: 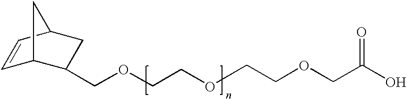
in which n represents an integer between approximately 50 and 340, 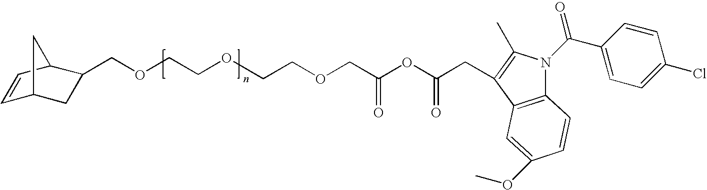
in which n represents an integer between approximately 50 and 340, 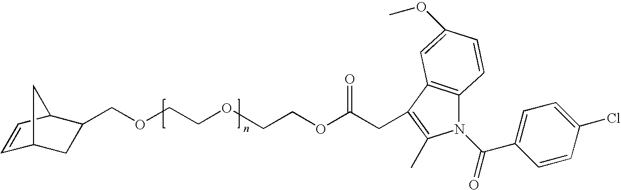
in which n represents an integer between approximately 50 and 340, 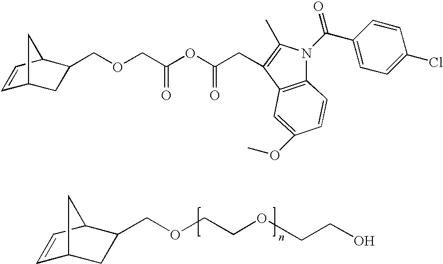
in which n represents an integer between approximately 50 and 340, 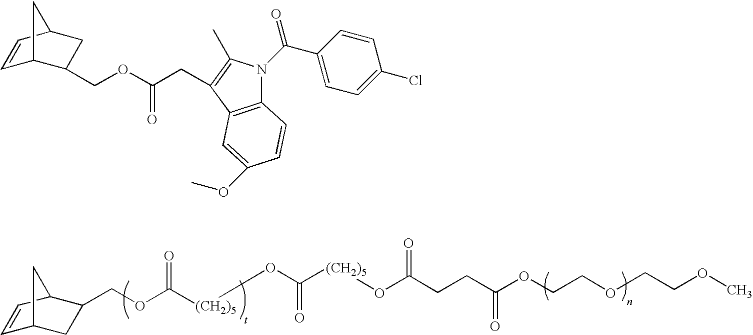
in which t represents an integer between 1 and 10 and n represents a whole number between approximately 50 and 340.

46. A method of preparation of spherical particles having a diameter between 10 nm and 100 μm, said particles being formed by polymer chains containing about 30 to 10000 monomer units, identical or different, derived from the polymerisation of monocyclic alkenes wherein the number of carbon atoms constituting the ring is of about 4 to 12 or polycyclic alkenes, wherein the total number of carbon atoms constituting the rings is of about 6 to 20, and wherein at least one of the said monomer units are substituted by a chain R comprising an ethylene polyoxide of formula (A) which are covalently bonded to the said monomer units via a hydrolysable bridge or not covalently bonded via such a hydrolysable bridge
(CH₂—CH₂—O)ₙ—X (A)
wherein n represents an integer from about 50 to 340, especially from 70 to 200, and X represents an alkyl or alkoxy chain with about 1 to 10 carbon atoms, comprising a reactive function of the OH, halogen, NH₂, C(O)X₁ type in which X₁ represents a hydrogen atom, a halogen atom, an OR′ or NHR′ group wherein R′ represents a hydrogen atom or a hydrocarbon chain with approximately 1 to 10 carbon atoms, substituted or unsubstituted, where X represents a polyene or another group comprising a photosensitive function, the said reactive function being optionally engaged in a bond with an active ingredient or a protein or another biological molecule, the said chain R being bonded covalently to the said monomers, which comprises making use of monocyclic or polycyclic alkenes according to claim 43.
